(19) United States
(12) Patent Application Publication
(10) Pub. No.: US 2015/0150923 A1
(43) Pub. Date: Jun. 4, 2015

(54) COMPOSITION FOR TREATMENT OF URINARY INCONTINENCE AND THE PREPARATION PROCESS THEREOF

(71) Applicant: Uchechi L. Eze, Springs, TX (US)

(72) Inventor: Uchechi L. Eze, Springs, TX (US)

(21) Appl. No.: 14/609,377

(22) Filed: Jan. 29, 2015

Related U.S. Application Data
(63) Continuation-in-part of application No. 13/924,287, filed on Jun. 21, 2013.

(51) Int. Cl. A61K 36/28; A61K 47/12; A61K 47/14; A61K 47/10; A61K 47/22; A61K 47/36; A61K 47/42; A61K 47/26; A61K 35/644; A61K 47/02

(52) U.S. Cl. CPC A61K 36/28; A61K 35/644; A61K 47/12; A61K 47/14; A61K 47/02; A61K 47/22; A61K 47/36; A61K 47/42; A61K 47/26; A61K 47/10

(57) ABSTRACT

A composition of matter having the capability of inhibiting Urinary Incontinence, comprising 0.5 to 40% of a first active agent comprising an extract of plant vernonia amygdaline; 0.5 to 40% of a second active agent comprising honey; 5% of a first solvent or first mixture of solvents selected from a group of acids consisting of Gluconic, Formic, Acetic, Autyric, Citric, Lactic, Malic, Pyroglutamic, Propionic, Valeric, Capronic, Palmitic, and Succinic; and 5% of a second solvent or second mixture of solvents selected from a group of excipients consisting of Stearin, Magnesium Stearate, Silica, Titanium dioxide, Indigotine, Maldodextrin, Stearic, Curculin, Erythritol, Glycyrrhizin, and Glycerol, Isomalt.

### FIELD OF THE INVENTION

The present invention relates to novel compositions for urinary incontinence treatment comprising at least one agent derived from a natural source as active agent in combination with at least one other active agent so that the natural characteristics of the first agent is changed and at least one excipient.

### BACKGROUND OF THE INVENTION

Techniques described herein relate generally to various compositions and methods of manufacturing of a composition for treatment of Urinary Incontinence.

Vernonia amygdaline plant, also known as rawa (Amharic), ewro (Yoruba), etidot (Ibibio), onugbu (Igbo), ityuna (Tiv), oriwo (Edo), chusar-doki (Hausa), muluuza (Luganda), labwori (Acholi), and olusia (Luo) is cultivated in Africa and grows frequently in that region.

Presently no literature has been found relating to the use of Vernonia amygdaline for the treatment of Urinary Incontinence. Urinary incontinence is a disorder wherein the patient experiences loss of bladder control due to age, prostate enlargement, or prostate cancer. Symptoms of said disorder can range from mild leaking to uncontrollable wetting and it can happen to anyone, but it becomes more common with age.

Therefore, a need exists for a continued search for novel natural products that may be used for treatment of Urinary Incontinence through natural remedies. The present invention not only provides a novel solution to the aforementioned problem, also describes various compositions comprising an extract of Onugbu in combination with at least one active agent for treatment of urinary incontinence.

### BRIEF SUMMARY OF THE INVENTION

This disclosure presents a composition for Urinary Incontinence treatment comprising at least one active ingredient; wherein the at least one active ingredient is derived from a natural source in combination with at least one other active ingredient; wherein the at least one other active ingredient is derived from a natural, artificial or semi-artificial source; and electively at least one excipient.

Furthermore, a process of making a composition for Urinary Incontinent treatment comprising steps of extraction from at least one active agent its essence; wherein the at least one active agent derived from a natural source; combination of at least one active agent with at least one other active agent; wherein at least one other active agent is derived from a natural, artificial or semi-artificial source; and addition optionally at least one excipient.

Furthermore, a method of treating Urinary Incontinence comprising: administrating to an individual a composition comprising at least one active agent; wherein the at least one active agent is derived from a natural source in combination with at least one other active agent; wherein the at least one other active agent is derived from a natural, artificial or semi-artificial source; and electively at least one excipient.

### DETAILED DESCRIPTION OF THE INVENTION

In the present invention, a composition for treatment of Urinary Incontinence is produced. The composition may comprise of at least two active agents. The active agents of the composition comprise of a first active agent extractable from a natural source. The first agent may be the essence of Vernonia amygdaline Plant (“Onugbu essence”). The at least second active agent may be an ingredient extracted from natural source or it may be an ingredient from an artificial source. The second agent and its characteristics is discussed below.

The essence of Vernonia amygdaline plant may be extracted from its leaves, seeds, stems or a combination thereof. Further, various processes may be employed to extract the essence of the plant. For example, by placing the plant under pressure, the essence may be extracted.

Also, heating the plant to vaporize its internal fluid and subsequently capturing the evaporated fluid through a condensing process may be employed to extract the plant's internal fluid. Further, the extraction of the essence of Vernonia amygdaline plant may be carried out with a polar solvent applicable for extraction. Leaves are first extracted with a polar solvent optionally by several times. The obtained solution is then mixed and extracted with a non-polar solvent to remove the waxes, essential oils, pigments and most of the non-polar molecules.

Furthermore, the second active agent may be obtained from a natural source. For example, the active ingredient may be honey, wherein honey can be procured from many sources including artificial, semi-artificial and natural sources. Conventionally, honey has been used as a disinfectant and topically used to heal wounds. It is widely perceived to possess antiseptic and antibacterial properties. The compositions of honey shows it has high level of monosaccharides, fructose and glucose, containing 70 to 80 percent sugar, giving it sweet taste, while the rest of its composition is made of minerals and water.

Honey may be mixed with the essence of the Onugbu essence t to produce the Urinary Incontinence Composition. Here, honey may be combined with the Onugbu essence to give the composition, not only a flavor, but also increased potency and effectiveness.

In addition, the composition may comprise a third active agent. For example, the third active agent may be one or more pharmaceutically acceptable excipients selected from but not limited to the group comprising diluents, disintegrants, binders, anti-adherants, glidants, anti-oxidants, buffering agents, colorants, flavoring agents, coating agents, solvents, viscosifying agents, waxes, wetting agents, emulsifying agents, solubilizers, stabilizers, buffering agents, vehicles, preservatives, surfactants, deodorants, colorants, stearin, magnesium stearate, stearic acid, Curculin, Erythritol, Glycyrrhizin, Glycerol, Isomalt, Stearin, Magnesium Stearate, Silica, Titanium dioxide, Indigotine, Maldodextrin, Stearic, Curculin, Erythritol, Glycyrrhizin, and Glycerol, Isomalt and the like.

In some embodiments of this invention acids such as Gluconic, formic, acetic, butyric, citric, lactic, malic, pyroglutamic, propionic, valeric, capronic, palmitic, and succinic may be added to increase the effectiveness and lasting duration of the composition in individuals.

In the present invention, the first and the second active agents may be having equal part weights in the composition. A range wherein one of the active ingredients has less than half part weight of the other active ingredient to where both active ingredients have equal part weights in the composition is considered. The effectiveness of the composition on an individual with Urinary Incontinence may vary by changing the ratio of the first agent's (e.g., Onugbu extract) part weight to the second agent's (e.g., honey) part weight in the composition. The third active agent may be added as desired to the composition.

In an exemplary embodiment, the composition may comprise 20 percent Onugbu essence and 80 percent honey, in one composition may comprise 50 percent Onugbu essence and 50 honey, or one composition may comprise 70 percent Onugbu essence and 30 percent honey. As the percentage of Onugbu essence in the composition increases, the composition remedial effect may last longer in a subject. As stated before, these combinations are exemplary and other ratios may be used to change the potency and effectiveness of the solution.

As discussed below, testing the composition on fifty-three (53) individuals who had urinary incontinence disorder showed that addition of honey to the essence increase the potency and duration of effectiveness of the composition in comparison to administrating the Onugbu essence alone to said individuals. Those test were performed for a period of three years.

It has been observed that the composition having equal Onugbu essence and honey part weights may treat the symptoms of Urinary Incontinence for four days in an individual with Urinary Incontinence. Further, when administrating the composition to a subject having Urinary Incontinence, wherein the Onugbu essence's part weight was half of the part weight of honey, the symptoms of Urinary Incontinence may be treated for two days. The composition may be administered to an individual with Urinary Incontinence in various forms including but not limit to liquid, cream, gel, lotion, spray, shake, capsule or pill.

It is further the process of this invention to extract from at least one active ingredient its essence; wherein the at least one active ingredient derived from a natural source. Here the natural source is the Vernonia amygdaline plant. Furthermore, the process includes combining the essence of the Vernonia amygdaline plant with at least one other active ingredient; wherein the at least one other active ingredient is derived from a natural, artificial or semi-artificial source. The other active ingredient may be honey, wherein it can be produced from a natural, artificial or semi-artificial source.

The process may also include electively adding of at least one excipient. The excipient may be one of excipient selected from a group comprising diluents, disintegrants, binders, anti-adherants, glidants, lubricants, antioxidants, buffering ingredients, colorants, flavoring ingredients, coating ingredients, solvents, osmotic ingredients viscosifymg ingredients, waxes, wetting ingredients, emulsifying ingredients, solubilizers, stabilizers, buffering ingredients, chelating ingredients, vehicles, preservatives, surfactants, deodorants, colorants, bulking ingredients, hydrophilic polymers, tonicity adjusting ingredients, local anesthetics, pH adjusting ingredients acids, sugar alcohol, reducing sugars and non-reducing sugars, or combination thereof.

For the preparation of the composition, varying volumes of excipients benzoic acid, sodium benzoate, 2-amino-1-(4-hydroxy-2-methanesulphonamidophenyl)ethanol, Isoflovane Duloxentine hydrochloride, Tolterodine, oxybutinine, any diisopropylamine derivative, amorphous flibanserin, pimavanserin, pioglitazone hydrochloride, benzalkoneum chlorides are taken in the compositions of increasing proportion in the various increments. For example, multiples of 5 (e.g., 5 ml, 10 ml, 15 ml, 20 ml, 25 ml, etc.,) has been used in preparing the composition. The temperature of the process is maintained between 30 and 70 degrees of Fahrenheit for varying temperatures. The vapors thus obtained contain vapors of volatile flavors of the Onugbu essence, the unevaporated portion is separated from the vapors and concentrated in the higher pressures with spray drying, and this mixture is added to different volumes of composition and biocompatible micro particles of tramadol. Some excipients are cell adhesion promotes. For example, tramadol may be used to absorb the volatile flavors of the composition and the flavor is substantially changed. Hence the result is an entirely new product altogether.

The discussion is regarding the amount of the solution each individual consumed and proportion and composition of ingredients used in that solution. Further, it shows the duration that each patient was treated after administration of the solution. The effect is compared to that of the solution when administered to individual with Urinary Incontinence with the effect of administrating only the essence of the Vernonia amygdaline plant. Each experience shows that honey increases the potency of the Onugbu essence. By adding honey to the Onugbu essence there has been a substantial increase in potency, taste as well as effectiveness. Further, addition of honey will also prevent any fermentation of the mixture and it helps stay indefinitely without deterioration. When the essence of the Vernonia amygdaline plant is added to a certain composition of sugar for a study, there has not been any substantial increase or decrease in the effectiveness of the solution.

With the addition of preservatives such as benzoic acid, sodium benzoate (“excipient”), and the salt form of tramadol (preferably tramadol hydrochloride) the organic composition seems to stay off undesirable chemical changes or decomposing microbial growth. Contained compositions of the excipients held desirable effect going by the studies on men and women. When there was a variation in measure and nature excipients, for example, instead if tramadol or benzoic acid, when other hydrophobic excipients, optical isomeric compounds or Isoflovane compounds were used, the study yielded the final product, which still had a healing effect on Urinary Incontinence in the individuals.

The above studies were carried out on a group size of about 50 in number with about 3-4 individuals per batch. The combinations of Essence, excipients and honey were administered to each of these batches in a designated temperatures to understand the effect of substance over a period of 3-4 days. After a certain number of days in each batch, the symptoms of Urinary Incontinence began to steadily disappear and gradually, there has been development in the condition of individuals.

The studies after administering these varying volumes of “Solution” brought different changes in different individuals. Each individual who is the subject of observation and study here is administered a certain volume of the mixture which consists of Onugbu essence, honey and excipient. The differing volumes of the substance help us understand the effect of excipient in combination with honey and Onugbu essence. Once the different sets of people are divided into various batches for study, the mixture is administered in the volume of 5 ml upwards.

These volumes of administered mixture have a certain effect of individuals over the next few days. In few instances, changes were noticed in a matter of 24 hours. In other cases, it took three to four days. This administering of volume and physical conditions of the people under study is duly recorded and tabulated in the applicant's Administration Affidavit incorporated herein by reference.

The administration process further revealed that while few saw rashes disappear, few says persistent relief over a stretch of four to five days with no signs of urinary incontinence. Almost all the participants reported no side effects of the administered product.

In some embodiments, the composition of the present matter inhibits Urinary Incontinence. In those composition, 0.5 to 40% of a first active agent which is an extract of plant vernonia amygdaline is present. Furthermore, 0.5 to 40% of a second active agent which is honey is added as well. The composition will require 5% of a first solvent or first mixture of solvents selected from a group of acids consisting of Gluconic, Formic, Acetic, Autyric, Citric, Lactic, Malic, Pyroglutamic, Propionic, Valeric, Capronic, Palmitic, and Succinic. The acid facilitate reaction between the molecules of honey and the extract of plant vernonia amygdaline. Furthermore, 5% of a second solvent or second mixture of solvents selected from a group of excipients consisting of Stearin, Magnesium Stearate, Silica, Titanium dioxide, Indigotine, Maldodextrin, Stearic, Curculin, Erythritol, Glycyrrhizin, and Glycerol, Isomalt is added to complete the composition. The second solvent acts to preserve the composition.

In other embodiments of the composition, by weight, may consist of 5% to 25% of the first active agent, 5% to 65% of the second active agent, 5% of the first solvent or first mixture of solvents; and 5% of the second solvent or second mixture of solvents.

Here, the extract of plant Vernonia amygdaline is obtained by rinsing a suitable amount of plant Vernonia amygdaline with clean water for a suitable period of time. Furthermore, the suitable amount of plant Vernonia amygdaline after the rinsing should be placed in a suitable boiler apparatus. A suitable amount of water should be added to the boiler apparatus to cover the suitable amount of plant Vernonia amygdaline. Furthermore, the suitable amount of plant Vernonia amygdaline in the suitable boiler apparatus must be boiled for a suitable period of time. The extract after boiling must be filtered to remove particulate matter (e.g., the remain of the plant). That amounts and measurements are not arbitrary, rather the ratios must be consistent. As the ratio of extract with respect to honey changes the potency of the composition. In another words, more extract means more potency. However, addition of honey is critical because it makes the extract to last longer in an individual.

Aforementioned composition is to be administered orally to a patient having Urinary Incontinence for a suitable period of time. Example of those periods are hourly, daily, or weekly.

Although no efforts have been made to commercialize the product and optimize it to scale it up to industrial applicability, through the control of special operations, the novel combination with various compositions embodying the present invention can be achieved. It is the process of making the essence capable of manufacturing a product possessing the healing characteristics of a manufactured drug in artificially administered conditions that makes this invention an exception.

1. A composition of matter having the capability of inhibiting Urinary Incontinence, comprising:
(a) 0.5 to 40% of a first active agent comprising an extract of plant vernonia amygdaline;
(b) 0.5 to 40% of a second active agent comprising honey;
(c) 5% of a first solvent or first mixture of solvents selected from a group of acids consisting of Gluconic, Formic, Acetic, Autyric, Citric, Lactic, Malic, Pyroglutamic, Propionic, Valeric, Capronic, Palmitic, and Succinic; and
(d) 5% of a second solvent or second mixture of solvents selected from a group of excipients consisting of Stearin, Magnesium Stearate, Silica, Titanium dioxide, Indigotine, Maldodextrin, Stearic, Curculin, Erythritol, Glycyrrhizin, and Glycerol, Isomalt.

2. The composition according to claim 1, comprising, by weight, from 5% to 25% of the first active agent, 5% to 65% of the second active agent comprising honey, 5% of a first solvent or first mixture of solvents; and 5% of a second solvent or second mixture of solvents.

3. The composition according to claim 1, wherein the extract of plant Vernonia amygdaline is obtained by rinsing a suitable amount of plant Vernonia amygdaline with clean water for a suitable period of time, placing a suitable amount of plant Vernonia amygdaline after the rinsing in a suitable boiler apparatus, adding a suitable amount of water to cover the suitable amount of plant Vernonia amygdaline, boiling the suitable amount of plant Vernonia amygdaline in the suitable boiler apparatus for a suitable period of time, and filtering the resulting extract to remove particulate matter.

4. A method of administering a composition comprising:
administering separately suitable dosages orally of the composition to a patient having Urinary Incontinence for a suitable period of time, the composition is prepared by combining 0.5 to 40% of a first active agent comprising an extract of plant Vernonia amygdaline, wherein the extract is obtained by rinsing a suitable amount of plant Vernonia amygdaline with clean water for a suitable period of time, placing a suitable amount of plant Vernonia amygdaline after the rinsing in a suitable boiler apparatus, adding a suitable amount of water to cover the suitable amount of plant Vernonia amygdaline, boiling the suitable amount of plant Vernonia amygdaline in the suitable boiler apparatus for a suitable period of time, and filtering the resulting extract to remove particulate matter; with 0.5 to 40% of a second active agent comprising honey; 5% of a first solvent or first mixture of solvents selected from a group of acids consisting of Gluconic, Formic, Acetic, Autyric, Citric, Lactic, Malic, Pyroglutamic, Propionic, Valeric, Capronic, Palmitic, and Succinic; and 5% of a second solvent or second mixture of solvents selected from a group of excipients consisting of Stearin, Magnesium Stearate, Silica, Titanium dioxide, Indigotine, Maldodextrin, Stearic, Curculin, Erythritol, Glycyrrhizin, and Glycerol, Isomalt.
